(19) United States
(12) Patent Application Publication
(10) Pub. No.: US 2022/0226143 A1
(43) Pub. Date: Jul. 21, 2022

(54) Ostomy Garment Assembly

(71) Applicant: Daniel F. Negrete, Crown Point, IN (US)

(72) Inventor: Daniel F. Negrete, Crown Point, IN (US)

(21) Appl. No.: 17/151,736

(22) Filed: Jan. 19, 2021

(51) Int. Cl. A61F 5/449

(52) U.S. Cl. CPC A61F 5/449

(57) ABSTRACT

An ostomy garment assembly for securing and concealing an ostomy bag includes a belt that is wearable around a user's waist. The belt has an opening extending therethrough to accommodate an ostomy bag that is being worn by the user. A pouch is coupled to the belt for containing the ostomy bag being worn by the user. The pouch is comprised of an opaque material to conceal the ostomy bag. Additionally, the pouch is comprised of a fluid impermeable material to contain fluids that might leak from the ostomy bag. The pouch is openable without removing the pouch from the belt to facilitate the ostomy bag to be emptied while the ostomy bag is positioned within the pouch.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to ostomy devices and more particularly pertains to a new ostomy device for securing and concealing an ostomy bag. The ostomy device includes a belt that is wearable around the user's waist and a pouch that is coupled to the belt for receiving the ostomy bag. Additionally, the pouch can be opened for draining the ostomy bag without requiring the user to remove the belt or the pouch.

### (2) Description of Related Art Including Information Disclosed Under 37 CFR 1.97 and 1.98

The prior art relates to ostomy devices including a variety of undergarments that have a pouch integrated therein for storing an ostomy bag. The prior art discloses an ostomy support belt that has an opening therein and which facilitates an ostomy bag to be attached thereto.

### BRIEF SUMMARY OF THE INVENTION

An embodiment of the disclosure meets the needs presented above by generally comprising a belt that is wearable around a user's waist. The belt has an opening extending therethrough to accommodate an ostomy bag that is being worn by the user. A pouch is coupled to the belt for containing the ostomy bag being worn by the user. The pouch is comprised of an opaque material to conceal the ostomy bag. Additionally, the pouch is comprised of a fluid impermeable material to contain fluids that might leak from the ostomy bag. The pouch is openable without removing the pouch from the belt to facilitate the ostomy bag to be emptied while the ostomy bag is positioned within the pouch.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective phantom view of an ostomy garment assembly according to an embodiment of the disclosure.

FIG. 2 is a back perspective phantom view of an embodiment of the disclosure.

FIG. 3 is a bottom perspective view of an embodiment of the disclosure.

FIG. 4 is a front phantom view of an embodiment of the disclosure.

FIG. 5 is a perspective in-use view of an embodiment of the disclosure.

FIG. 6 is a front phantom view of an alternative embodiment of the disclosure.

### DETAILED DESCRIPTION OF THE INVENTION

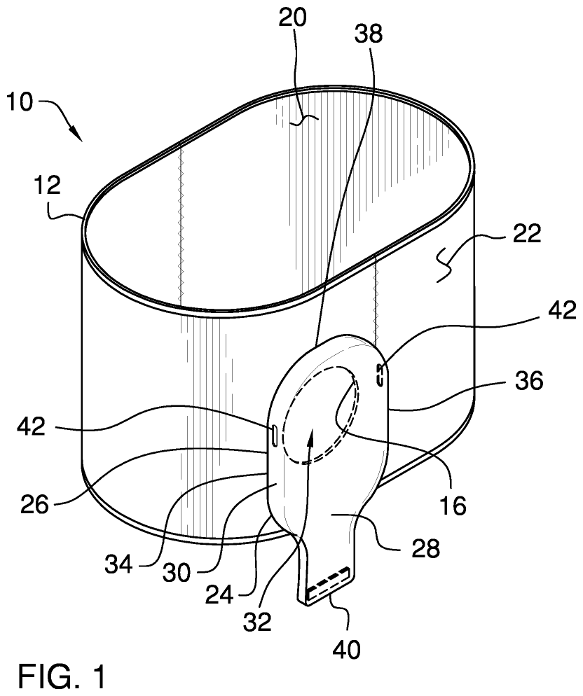

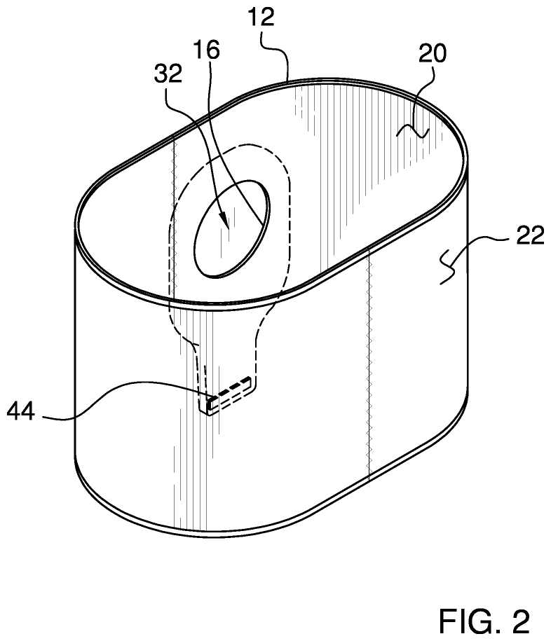

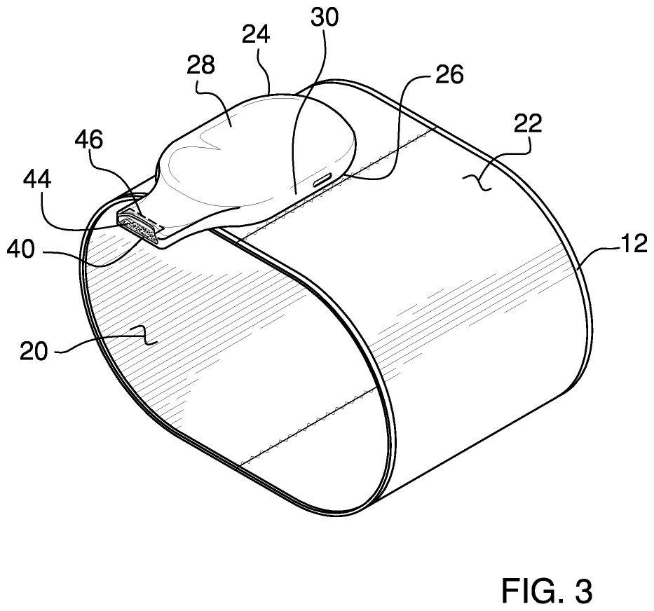

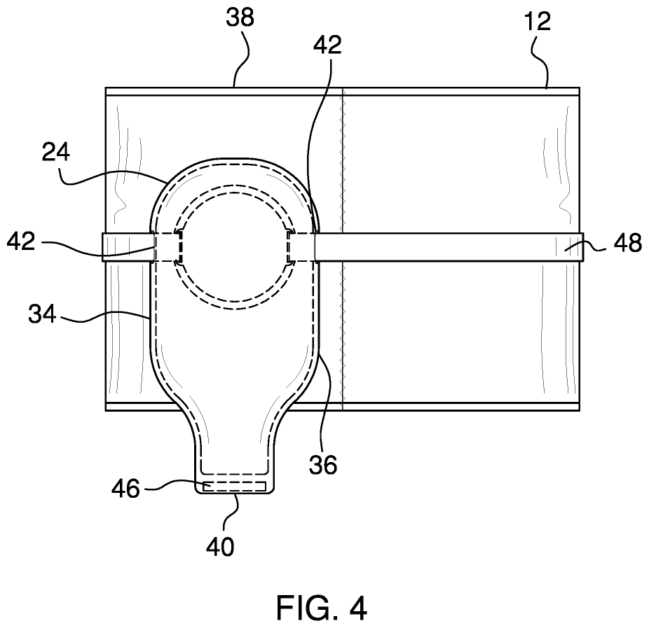

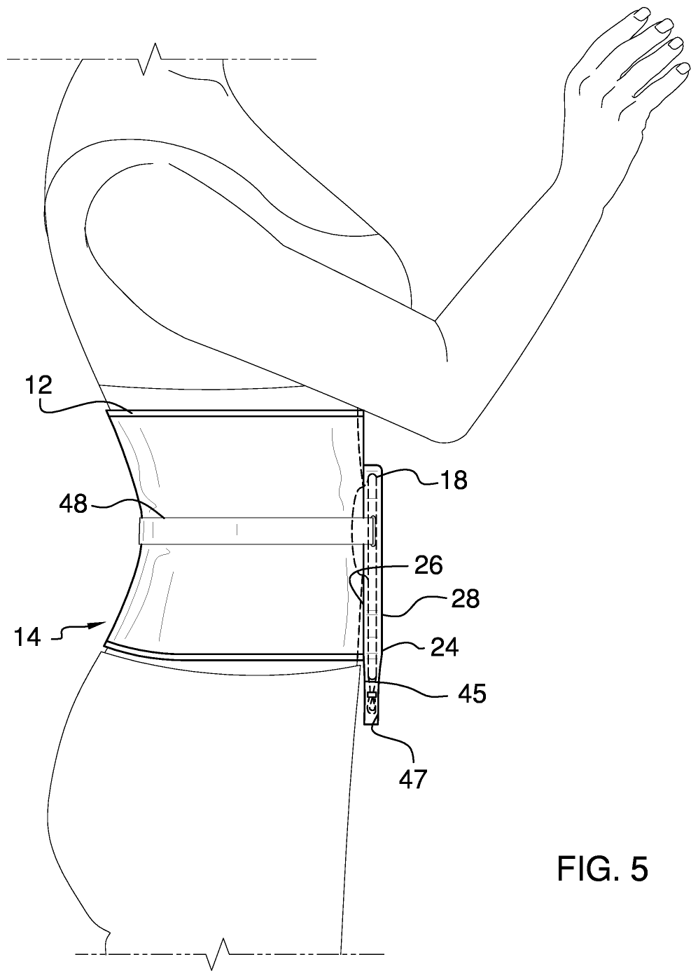

With reference now to the drawings, and in particular to FIGS. 1 through 6 thereof, a new ostomy device embodying the principles and concepts of an embodiment of the disclosure and generally designated by the reference numeral 10 will be described.

As best illustrated in FIGS. 1 through 6, the ostomy garment assembly 10 generally comprises a belt 12 that is wearable around a user's waist 14. The belt 12 is comprised of a resiliently stretchable material such that the belt 12 compresses around the user's waist 14. Additionally, the belt 12 has an opening 16 extending therethrough to accommodate an ostomy bag 18 being worn by the user. The belt 12 has an inside surface 20 and an outside surface 22, and the opening 16 extends through the inside surface 20 and the outside surface 22.

A pouch 24 is coupled to the belt 12 and the pouch 24 can contain the ostomy bag 18 being worn by the user. The pouch 24 is comprised of an opaque material to conceal the ostomy bag 18. In this way the user can rest assured that the ostomy bag 18 will not be visible to observers. The pouch 24 is comprised of a fluid impermeable material to contain fluids that might leak from the ostomy bag 18. In this way the pouch 24 and the belt 12 facilitate the ostomy bag 18 to be discretely supported without the risk of unsightly leaking.

The pouch 24 has a back wall 26, a front wall 28 and an outer wall 30 extending therebetween, and the back wall 26 has a hole 32 extending into an interior of the pouch 24. The back wall 26 is attached to the outside surface 22 of the belt 12 having the hole 32 being aligned with the opening 16 in the belt 12. In this way the pouch 24 can receive the ostomy bag 18 when the belt 12 is worn around the users' waist 14. The outer wall 30 has a first lateral side 34, a second lateral side 36, an upper side 38 and a lower side 40. Each of the first lateral side 34 and the second lateral side 36 curves inwardly toward each other at a point that is located adjacent to the lower side 40 such that the lower side 40 has a width is less than the width of the upper side 38.

The lower side 40 is open to access an interior of the pouch 24 thereby facilitating the ostomy bag 18 to be emptied through the lower side 40. In this way the ostomy bag 18 can be emptied without the need to remove the ostomy bag 18 from the pouch 24. Each of the first lateral side 34 and the second lateral side 36 has a respective one of a pair of slots 42 extending into an interior of the pouch 24. Moreover, the slots 42 are aligned with each other.

A first mating member 44 is coupled to an inside surface 45 of the back wall 26 of the pouch 24, and the first mating member 44 is aligned with the lower side 40. A second mating member 46 is coupled to an inside surface 47 of the front wall 28 of the pouch 24, and the second mating member 46 is aligned with the lower side 40. The second mating member 46 releasably engages the first mating member 44 for closing the lower side 40 of the pouch 24.

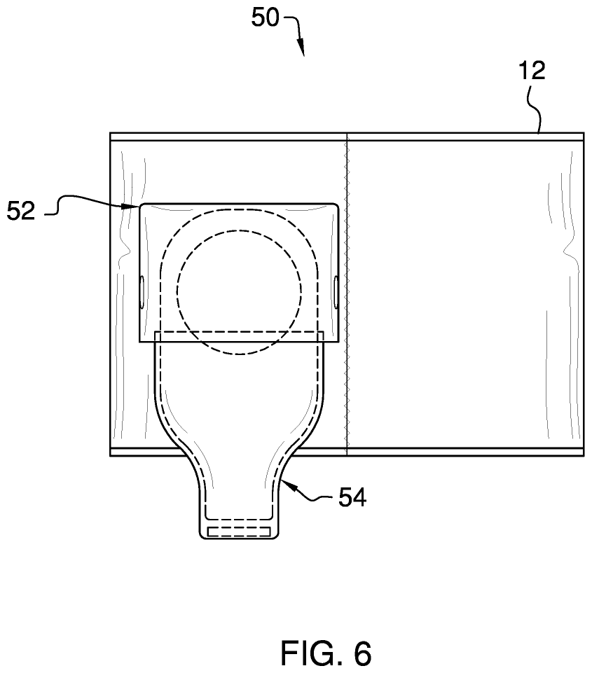

A strap 48 is extendable through each of the slots 42 in the first lateral side 34 and the second lateral side 36 of the outer wall 30 of the pouch 24. The strap 48 is extendable around the belt 12 for increasing stability of the belt 12 on the user. The strap 48 can be employed when the user is recovering from a hernia repair surgery to reduce strain on the user. In an alternative embodiment 50 as is most clearly shown in FIG. 6, the pouch 24 has a top portion 52 that is separate from a bottom portion 54. The top portion 52 is liftable away from the bottom portion 54 to access an interior of the pouch 24. In this way the ostomy bag 18 can be inserted into the pouch 24 or removed from the pouch 24.

In use, the belt 12 is worn around the user's waist 14 such that the user's ostomy bag 18 can be inserted through the opening 16 in the belt 12 and into the pouch 24. In this way the ostomy bag 18 can be supported and concealed to enhance the user's self confidence. Moreover, the ostomy bag 18 is inhibited from getting hung up and potentially dislodged or torn while the ostomy bag 18 is contained in the pouch 24. The ostomy bag 18 can be emptied through the lower side 40 of the pouch 24 without requiring the user to remove the belt 12 and the pouch 24.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An ostomy garment assembly for supporting an ostomy bag on a user, said assembly comprising:
a belt being wearable around a user's waist, said belt being comprised of a resiliently stretchable material wherein said belt is configured to compress around the user's waist, said belt having an opening extending therethrough wherein said opening is configured to accommodate an ostomy bag being worn by the user; and
a pouch being coupled to said belt wherein said pouch is configured to contain the ostomy bag being worn by the user, said pouch being comprised of an opaque material wherein said pouch is configured to conceal the ostomy bag, said pouch being comprised of a fluid impermeable material wherein said pouch is configured to contain fluids that might leak from the ostomy bag, said pouch being openable without removing said pouch from said belt wherein said pouch is configured to facilitate the ostomy bag to be emptied while the ostomy bag is positioned within said pouch.

2. The assembly according to claim 1, wherein:
said belt has an inside surface and an outside surface, said opening extending through said inside surface and said outside surface; and
said pouch has a back wall, a front wall and an outer wall extending therebetween, said back wall having a hole extending into an interior of said pouch, said back wall being attached to said outside surface of said belt having said hole being aligned with said opening in said belt wherein said pouch is configured to receive the ostomy bag.

3. The assembly according to claim 2, wherein said outer wall has a first lateral side, a second lateral side, an upper side and a lower side, each of said first lateral side and said second lateral side curving inwardly toward each other at a point being located adjacent to said lower side such that said lower side has a width being less than the width of said upper side.

4. The assembly according to claim 3, wherein said lower side is open to access an interior of said pouch wherein said lower side is configured to facilitate the ostomy bag to be emptied through said lower side.

5. The assembly according to claim 3, wherein each of said first lateral side and said second lateral side has a respective one of a pair of slots extending into an interior of said pouch, said slots being aligned with each other.

6. The assembly according to claim 4, further comprising:
a first mating member being coupled to an inside surface of said back wall of said pouch, said first mating member being aligned with said lower side; and
a second mating member being coupled to an inside surface of said front wall of said pouch, said second mating member being aligned with said lower side, said second mating member releasably engaging said first mating member for closing said lower side of said pouch.

7. The assembly according to claim 5, further comprising a strap being extendable through each of said slots in said first lateral side and said second lateral side of said outer wall of said pouch, said strap being extendable around said belt for increasing stability of said belt on the user.

8. An ostomy garment assembly for supporting an ostomy bag on a user, said assembly comprising:
a belt being wearable around a user's waist, said belt being comprised of a resiliently stretchable material wherein said belt is configured to compress around the user's waist, said belt having an opening extending therethrough wherein said opening is configured to accommodate an ostomy bag being worn by the user, said belt having an inside surface and an outside surface, said opening extending through said inside surface and said outside surface;
a pouch being coupled to said belt wherein said pouch is configured to contain the ostomy bag being worn by the user, said pouch being comprised of an opaque material wherein said pouch is configured to conceal the ostomy bag, said pouch being comprised of a fluid impermeable material wherein said pouch is configured to contain fluids that might leak from the ostomy bag, said pouch having a back wall, a front wall and an outer wall extending therebetween, said back wall having a hole extending into an interior of said pouch, said back wall being attached to said outside surface of said belt having said hole being aligned with said opening in said belt wherein said pouch is configured to receive the ostomy bag, said outer wall having a first lateral side, a second lateral side, an upper side and a lower side, each of said first lateral side and said second lateral side curving inwardly toward each other at a point being located adjacent to said lower side such that said lower side has a width being less than the width of said upper side, said lower side being open to access an interior of said pouch wherein said lower side is configured to facilitate the ostomy bag to be emptied through said lower side, each of said first lateral side and said second lateral side having a respective one of a pair of slots extending into an interior of said pouch, said slots being aligned with each other;
a first mating member being coupled to an inside surface of said back wall of said pouch, said first mating member being aligned with said lower side;
a second mating member being coupled to an inside surface of said front wall of said pouch, said second mating member being aligned with said lower side, said second mating member releasably engaging said first mating member for closing said lower side of said pouch; and
a strap being extendable through each of said slots in said first lateral side and said second lateral side of said outer wall of said pouch, said strap being extendable around said belt for increasing stability of said belt on the user.

9. The assembly according to claim 8, wherein said pouch has a top portion being separate from a bottom portion, said top portion being liftable away from said bottom portion to access an interior of said pouch wherein said top portion is configured to facilitate the ostomy bag to be inserted into or removed from said pouch.
